(19) United States
(12) Patent Application Publication
(10) Pub. No.: US 2021/0038564 A1
(43) Pub. Date: Feb. 11, 2021

(54) METHODS FOR SCAR PREVENTION

(71) Applicant: Northwestern University, Evanston, IL (US)

(72) Inventors: Thomas A. Mustoe, Chicago, IL (US); Peter Kim, Chicago, IL (US); Jason Ko, Chicago, IL (US); Xianzhong Ding, Chicago, IL (US); Yanan Zhao, Chicago, IL (US)

(21) Appl. No.: 16/999,823

(22) Filed: Aug. 21, 2020

Related U.S. Application Data
(63) Continuation of application No. 15/407,747, filed on Jan. 17, 2017.
Application No. 15/407,747 is a continuation of application No. 12/906,719, filed on Oct. 18, 2010, now abandoned.
(60) Provisional application No. 61/252,538, filed on Oct. 16, 2009.

(51) Int. Cl. A61K 31/366; A61K 31/522; A61K 31/56; A61K 36/8962; A61K 31/436; A61K 31/25; A61K 38/18; A61K 31/513; A61K 31/704; A61K 31/397; A61K 9/00; A61K 31/22; A61K 45/06

(52) U.S. Cl. CPC A61K 31/366; A61K 31/522; A61K 31/56; A61K 36/8962; A61K 31/436; A61K 31/25; A61N 5/0616; A61K 31/513; A61K 31/704; A61K 31/397; A61K 9/0019; A61K 31/22; A61K 45/06; A61K 38/1841

(57) ABSTRACT

Provided herein are compositions and methods for preventing or reducing scar formation (e.g., hypertrophic scars). Certain embodiments provide a method of preventing hypertrophic scar formation in a subject comprising administering a HMG-CoA reductase-inhibiting agent to a wound site. In some embodiments, the wound site comprises scar tissue.

### CROSS REFERENCE TO RELATED APPLICATIONS

This application is a continuation of U.S. patent application Ser. No. 15/407,747, filed Jan. 17, 2017, which is a continuation of U.S. patent application Ser. No. 12/906,719, filed Oct. 18, 2010, now abandoned, which claims priority to U.S. Provisional Application Ser. No. 61/252,538, filed Oct. 16, 2009, each of which are herein incorporated by reference in its entirety.

### FIELD OF THE INVENTION

Provided herein are compositions and methods for preventing or reducing scar formation (e.g., hypertrophic scars). For example, provided herein are methods of administrating HMG-CoA reductase-inhibiting agents for preventing or reducing scar formation.

### BACKGROUND

When a wound heals, a scar takes its place. Simple tissues such as fat, connective tissue, and epithelium regenerate, but the skin, being a complex organ derived from two germ layers, heals by the formation of a predominantly fibrous tissue, i.e., a scar. If the injury sections or destroys the papillary layer of the stratum corneum, a scar will always be formed. Sometimes, this scar is inconspicuous; other times, it may be disfiguring.

Examples of disfiguring scars include keloids, widened scars, and hypertrophic scars. Both keloid and hypertrophic scars are wounds that heal overzealously above the skin surface. The difference between a keloid and a hypertrophied scar is that a keloid continues to enlarge beyond the original size and shape of the wound, while a hypertrophic scar enlarges within the confines of the original wound. Hypertropic scars often lead to tightening or shortening of the skin and/or underlying muscles. This type of scar is associated with adverse wound healing factors. Hypertrophic scars may occur in persons of any age or at any site, and skin tension is frequently implicated in hypertrophic scar formation. Both keloids and hypertrophic scars can recur after surgical excision; however, the recurrence of keloid scars is more common. Widened scars are wounds that separate during the healing process, usually in response to tension perpendicular to the wound edges.

Hypertrophic scars can be aesthetically displeasing and can impair function in a location-dependent manner (e.g., when located over joints). Contracture over certain muscles can be debilitating, especially in the face. Treatment for hypertrophic scars, however, is limited. Aside from mechanical treatments (e.g., occlusive dressings, compression therapy), administration of silicone gel to promote wound hydration, and/or administration of steroids to the wound site, there are few treatments available. Scar response to treatment varies and some treatment methods (e.g., topical steroid administration) are contraindicated for certain classes of patients (e.g., patients with bacterial infections, yeast infections, or viral infections affecting the wound site). Accordingly, improved methods for treating abnormal scars (e.g., hypertrophic scars) are needed.

### SUMMARY

Provided herein are compositions and methods for preventing or reducing scar formation (e.g., hypertrophic scars). For example, provided herein are methods of administrating HMG-CoA reductase-inhibiting agents for preventing or reducing scar formation.

Abnormal scars include hypertrophic scars, keloids, and widened wounds. Hypertrophic scars form when scar tissue elevates above the surrounding non-wound tissue, but the scar does not extend laterally beyond the original boundaries of the wound. In addition to causing cosmetic or aesthetic concern, hypertrophic scars can limit range of motion (e.g., when located over a joint or certain musculature, such as the musculature of the face) and can cause pain, burning sensation, and/or puritic sensation. In experiments conducted during the course of developing some embodiments provided herein, it was surprisingly found that locally administered HMG-CoA reductase inhibitors (e.g., statins) inhibit hypertrophic scar formation (e.g, reduce scar elevation index). The potential clinical importance of these findings is significant. Efforts to minimize scarring, whether physiologic or pathologic (e.g., hypertrophic scar), are constantly made in numerous clinical situations, be it traumatic (e.g., burn) or post-operative scarring that is in question. The ability to minimize scarring on a consistent basis, even marginally, has very large clinical ramifications, and significant research and funding resources have been put toward investigating various means of minimizing, or eliminating, scarring.

Statins (HMG-CoA reductase inhibitors), as a class of medications, are the most popular and heavily prescribed medications for hyperlipidemia. The number of people taking statins is impressive and ever-growing, especially in the US. Experiments conducted during the course of developing the present technology show a link between statins and reduced scarring. The fact that minimal scarring is one of the ideal goals of every surgeon, plastic surgeon or not, and that statins—the most commonly prescribed anti-lipid therapy in the world—may effectively reduce scarring, is a monumental finding with innumerable clinical ramifications.

The methods provided herein are not limited by the nature of the HMG-CoA reductase-inhibiting agent. In preferred embodiments, the HMG-CoA reductase-inhibiting agent is a statin. Statins include, but are not limited to, Atorvastatin (brand names Lipitor, Torvast), Cerivastatin (brand names Lipobay, Baycol), Fluvastatin (brand names Lescol, Lecol XL), Lovastatin (brand names Mevacor, altocor, Altoprey), Mevastatin (naturally occurring in organisms including, but not limited to, oyster mushrooms and Monascus purpureus), Pitavastatin (brand names Lovalo, Pitava), Pravastatin (brand names Pravachol, Selektine, Lipostat), Rosuvastatin (brand name Crestor), and Simvastatin (brand names Zocor, Lipex). Statins are often used in combination with other agents, including but not limited to, Simvastatin+Ezetimibe combination therapy (brand name Vytorin), Lovastatin+Niacin combination therapy (brand name Advicor), Atorystatin+Amlidipine combination therapy (brand name Caduet), and Simvastatin+Niacin combination therapy (brand name Simcor).

In some embodiments, local administration is achieved by injection (e.g., subcutaneous injection, intramuscular injection). In some embodiments, local administration is achieved by topical administration (e.g., by irrigation, by application of ointments, salves, powders, or the like.) In some embodiments, administration occurs proximal to the wound site. In some embodiments, administration occurs within the wound site. The methods are not limited by the nature of the wound. Wounds may occur via trauma, burns, surgical or other medical procedures, or as a result of a physiological condition (e.g., pathological conditions, pressure sores, ulcers).

The methods are not limited by temporal aspects of administering HMG-CoA reductase-inhibiting agents. Such agents may be administered before a wound is present, at the time of wound infliction, shortly after a wound is inflicted, or after a delay from the time that the wound was inflicted. Administration of the HMG-CoA reductase-inhibiting agent may occur less frequently than every 30 days, once every 7-30 days, once every 5-7 days, once every 4-5 days, once every 3-4 days, once every 2-3 days, once every 1-2 days, once a day, twice a day, three times a day, four times a day, or more than four times per day.

In certain embodiments, the present technology provides methods for lowering a scar elevation index in a subject comprising administering a HMG-CoA reductase-inhibiting agent to a wound site. In some embodiments, the wound site comprises scar tissue. In some embodiments, the wound site does not comprise scar tissue. In some embodiments, the HMG-CoA reductase-inhibiting agent comprises a statin. In some embodiments, the statin is an agent such as Atorvastatin, Cerivastatin, Fluvastatin, Lovastatin, Mevastatin, Pitavastatin, Pravastatin, Rosuvastatin, and Simvastatin. In some embodiments, administration occurs by injection. In some embodiments, the method further comprises administration of an agent such as corticosteroids, interferon, 5-fluorouracil, doxorubicin, bleomycin, verapamil, retinoic acid, imiquimod, tamoxifen, tacrolimus, botulinum toxin, onion extract, hydrocortisone, silicone, vitamin E, TGF-beta (TGF-beta1, TGF-beta2, TGF-beta3), VEGF inhibitors, etanercept, mannose-6-phosphate inhibitors, recombinant human interleukin-10, proline-cis-hydroxyproline, azetidine carboxylic acid, tranilast, pentoxifylline, anti-TGF agents (e.g., decorin), and Gentian violet. In some embodiments, the method further comprises administration of an additional treatment such as occlusive dressings, compression therapy, cryosurgery, excision, radiation therapy, laser therapy, photodynamic therapy, UVA-1 therapy, narrowband UVB therapy, and intense pulsed light therapy. In some embodiments, the HMG-CoA reductase-inhibiting agent is administered at a dose ranging from 1-500 μM per wound site. In some embodiments, the HMG-CoA reductase-inhibiting agent is administered at a dose of 0.1-1000 μg per wound site. In some embodiments, the scar elevation index is lowered by at least 5%. In some embodiments, the scar elevation index is lowered by at least 10%. In some embodiments, the scar elevation index is lowered by at least 20%. In some embodiments, the scar elevation index is lowered by at least 50%. In some embodiments, the administration occurs twice a day. In some embodiments, the administration occurs once a day. In some embodiments, the administration occurs once every two days. In some embodiments, the administration occurs once every three days. In some embodiments, the administration occurs once every four days. In some embodiments, the administration occurs once every five days. In some embodiments, the administration occurs once every week.

Certain embodiments provide a method of preventing hypertrophic scar formation in a subject comprising administering a HMG-CoA reductase-inhibiting agent to a wound site. In some embodiments, the wound site comprises scar tissue. In some embodiments, the wound site does not comprise scar tissue.

Certain embodiments provide a method of locally inhibiting expression of connective tissue growth factor in epidermal tissue comprising administering a HMG-CoA reductase-inhibiting agent. In some embodiments, the administration occurs proximal to a wound site. In some embodiments, the administration occurs at a wound site. In some embodiments, the administration occurs by injection. In some embodiments, the HMG-CoA reductase-inhibiting agent is an agent such as Atorvastatin, Cerivastatin, Fluvastatin, Lovastatin, Mevastatin, Pitavastatin, Pravastatin, Rosuvastatin, and Simvastatin.

Certain embodiments herein provide a kit for treating or preventing scar formation, said kit comprising a HMG-CoA reductase-inhibiting agent, a delivery device (e.g., for topical administration), and instructions for use. In some embodiments, the delivery device comprises a device for injection. In some embodiments, the HMG-CoA reductase inhibiting agent is an agent such as Atorvastatin, Cerivastatin, Fluvastatin, Lovastatin, Mevastatin, Pitavastatin, Pravastatin, Rosuvastatin, and Simvastatin.

Certain embodiments herein provide a method for treating a subject comprising: administering a HMG-CoA reductase-inhibiting agent locally to a wound site. In certain embodiments, the present technology provides a method of preventing hypertrophic scar formation in a subject comprising administering a HMG-CoA reductase-inhibiting agent locally to a wound site. In some embodiments, the present technology provides a method of locally inhibiting expression of connective tissue growth factor in epidermal tissue comprising administering a HMG-CoA reductase-inhibiting agent locally to epidermal tissue.

Additional embodiments will be apparent to persons skilled in the relevant art based on the teachings contained herein.

### DESCRIPTION OF THE DRAWINGS

FIG. 1 shows regions involved in measuring a scar elevation index (SEI).

FIG. 2 shows the results of treating 10-mm rabbit ear wounds (n=2 rabbits, 6 wounds per ear, 24 wounds in total) with Simvastatin.

FIG. 3 shows injection patterns for administering Simvastatin, Lovastatin, or Pravastatin to a wound in a rabbit ear model.

FIGS. 4A and 4B show the effects of three different doses of Simvastatin on scar elevation index in a rabbit ear model.

FIGS. 5A and 5B show the effects of three different doses of Lovastatin on scar elevation index in a rabbit ear model.

FIGS. 6A and 6B show the effects of three different doses of Pravastatin on scar elevation index in a rabbit ear model.

FIG. 7 shows the effects of low-dose (40 μM) Simvastatin, Lovastatin, or Pravastatin on scar elevation index as compared to controls in a rabbit ear model.

FIGS. 8A and 8B show scar elevation index values for rabbit ear wounds treated with vehicle (low control) or left untreated.

FIG. 9 shows a decrease in CTGF mRNA expression for rabbit ear wounds treated with a low dose of Simvastatin.

### DEFINITIONS

To facilitate an understanding of the present technology, a number of terms and phrases are defined below:

As used herein, “a” or “an” or “the” can mean one or more than one. For example, “a” cell can mean one cell or a plurality of cells.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “wound” refers broadly to injuries to tissue including the skin and subcutaneous tissue initiated in different ways, for example, by surgery, (e.g., incision sites, open post-cancer resection wounds, including but not limited to, removal of melanoma and breast cancer, etc.), contained post-operative surgical wounds, pressure sores (e.g., from extended bed rest), and wounds induced by trauma. As used herein, the term “wound” is used without limitation to the cause of the wound, be it a physical cause such as bodily positioning as in bed sores, impact as with trauma, or a biological cause such as disease process, aging process, obstetric process, or any other manner of biological process. Wounds caused by pressure may also be classified into one of four grades depending on the depth of the wound: Grade I wounds are limited to the epidermis; Grade II wounds extend into the dermis; Grade III wounds extend into the subcutaneous tissue; and Grade IV wounds expose bone (e.g., a bony pressure point such as the greater trochanter or the sacrum). The term “partial thickness wound” refers to wounds that are limited to the epidermis and dermis; a wound of any etiology may be partial thickness. The term “full thickness wound” is meant to include wounds that extend through the dermis.

As used herein, “wound site” refers broadly to the anatomical location of a wound, without limitation.

As used herein, the term “chronic wound” refers to a wound that has not healed within 30 days.

As used herein, the term “dressing” refers broadly to any material applied to a wound for protection, absorbance, drainage, treatment, etc. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) (Kannon and Garrett (1995) Dermatol. Surg. 21: 583-590; Davies (1983) Burns 10: 94; both of which are herein incorporated by reference). The present methods also contemplate the use of dressings impregnated with pharmacological compounds (e.g., antibiotics, antiseptics, thrombin, analgesic compounds, etc.). Cellular wound dressings include commercially available materials such as Apligraf®, Dermagraft®, Biobrane®, TransCyte®, Integra® Dermal Regeneration Template®, and OrCell®.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

As used herein, the term “sample” is used in its broadest sense. In one sense, it is meant to include a specimen or culture obtained from any source, as well as any biological. Biological samples may be obtained from animals (including humans) and encompass fluids, solids, tissues, and gases. Biological samples include blood products, such as plasma, serum and the like. Such examples are not however to be construed as limiting the sample types applicable to the present technology.

As used herein, the term “effective amount” refers to the amount of a compound (e.g., a wound-active compound as described herein) sufficient to effect beneficial or desired results. An effective amount can be administered in one or more administrations, applications, or dosages and is not limited to or intended to be limited to a particular formulation or administration route.

As used herein, the term “co-administration” refers to the administration of at least two agents (e.g., a statin and another wound-active agent as described herein) or therapies to a subject. In some embodiments, the co-administration of two or more agents or therapies is concurrent. In other embodiments, a first agent or therapy is administered prior to a second agent or therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a known potentially harmful (e.g., toxic) agents.

As used herein, the term “pharmaceutical composition” refers to the combination of an active agent with a carrier, inert or active, making the composition especially suitable for diagnostic or therapeutic use in vivo, in vitro, or ex vivo.

As used herein, the term “pharmaceutically acceptable carrier” refers to any of the standard pharmaceutical carriers, such as a phosphate buffered saline solution, water, emulsions (e.g., such as oil/water or water/oil emulsions), and various types of wetting agents. The compositions also can include stabilizers and preservatives. For examples of carriers, stabilizers, and adjuvants, see, e.g., Martin, Remington's Pharmaceutical Sciences, 15th Ed., Mack Publ. Co., Easton, Pa. (1975).

As used herein, the term “instructions for administering said compound to a subject,” and grammatical equivalents thereof, includes instructions for using the compositions contained in a kit for the treatment or prevention of scar formation (e.g., providing dosing, route of administration, decision trees for treating physicians for correlating patient-specific characteristics with therapeutic courses of action). The compositions of the present technology (e.g. as presented herein) can be packaged into a kit, which may include instructions for administering the compounds to a subject.

### DETAILED DESCRIPTION

Provided herein are compositions and methods for preventing or reducing scar formation (e.g., hypertrophic scars). For example, provided herein are methods of administrating HMG-CoA-inhibiting agents for preventing or reducing scar formation.

Adverse wound healing (e.g., scar formation, hypertropic scar formation, keloid formation, wound widening) can have long-term impact on the physical and psychological well-being of a patient, regardless of the nature and original cause of the wound (e.g, wounds due to trauma, surgery, burns, pressure wounds, ulcers). Hypertrophic scars are a common category of disfiguring scars. Hypertrophic scars lead to tightening or shortening of the skin and/or underlying muscles, causing both aesthetically displeasing long-term effects as well as impaired function when affecting movement of underlying joints or musculature.

While the present technology is not limited to any particular mechanism, and an understanding of the mechanism is not necessary to practice the present technology, it is contemplated that connective tissue growth factor (CTGF) is an autocrine growth factor that plays a significant role in wound healing (Sisco et al. (2008) Wound Repair Regen. 16:661-673; herein incorporated by reference in its entirety). CTGF is also known to play a role in fibrosis of the lung, kidney, and liver, acting downstream of TGF-β1 to facilitate cell proliferation, collagen deposition, angiogenesis, and fibroblast differentiation.

Simvastatin, Lovastatin, and Prevastatin are members of the statin class of drugs and are used clinically as cholesterol-lowering agents. These drugs act by inhibiting HMG-CoA reductase, the rate-limiting enzyme that catalyzes the conversion of HMG-CoA to mevalonate, a building block of cholesterol synthesis. Using in vitro models of pulmonary fibrosis, Simvastatin has been shown to inhibit both CTGF gene and protein expression in lung fibroblasts (Watts et al. (2005)Am. J Respir. Cell Mol. Biol. 32:290-300; Watts et al. (2006) Respir. Dis. 7:88-102; each herein incorporated by reference in its entirety). However, prior to the development of certain embodiments of the methods and compositions provided herein, the effect of statins on scar healing (e.g., inhibition of hypertrophic scar formation) was unknown. Surprisingly, experiments conducted during the course of developing some embodiments of the present technology have shown that statins delivered locally to a wound or surgical incision inhibit hypertrophic scar formation.

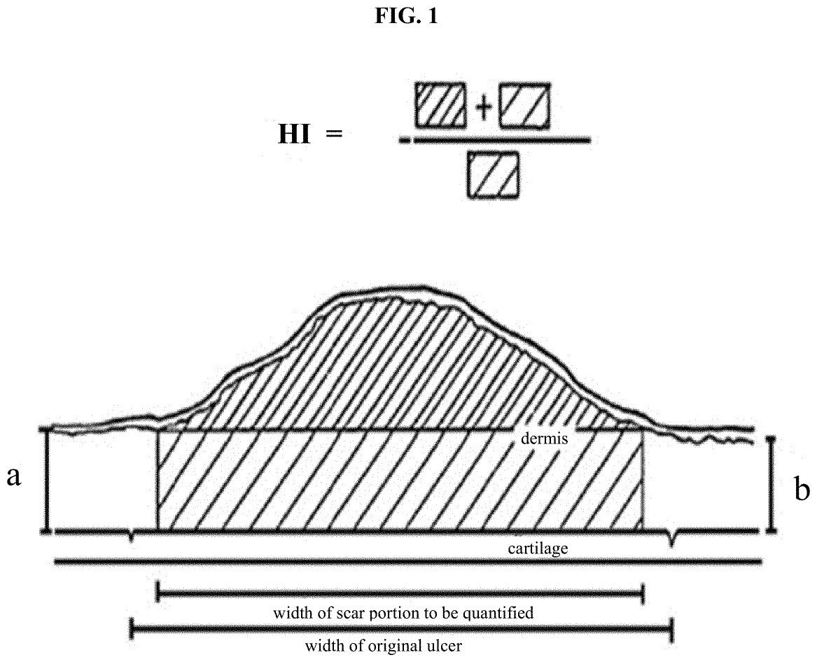

Using a well-established hypertrophic scar model in rabbit ear (Brown et al. (2008) Plast. Reconstr. Surg. 121:1165-1172; Kim et al. (2003) Wound Repair Regen. 11:368-372; Kryger et al. (2007) J Am. Coll. Surg. 205:78-88; Lu et al. (2005) J Am. Coll. Surg. 201:391-397; Morris et al. (2007) Plast. Reconstr. Surg. 100:674-681; Reid et al. (2006) Wound Repair Regen. 14:138-141; Reid et al. (2007) J Plast. Reconstr. Aesthet. Surg. 60:64-72; Saulis et al. (2002) Plast. Reconstr. Surg. 110:177-183; each herein incorporated by reference in its entirety), the effects of local administration of statins (e.g., Simvastatin, Lovastatin, Prevastatin) were investigated in experiments conducted during the course of developing some embodiments of the present technology. Multiple wounds of precise dimensions were made in rabbit ears, and statins were administered by injection within the wound site. The effectiveness of statin administration on inhibition of hypertrophic scar formation was assessed by histological determination of scar elevation index (FIG. 1). The scar elevation index is determined by the ratio of total wound area tissue height to the area of normal tissue below the hypertrophic scar. In this calculation, an SEI value of 1 is defined as a scar of equal height to the surrounding unwounded dermis, while an SEI value exceeding 1 is defined as a raised, hypertrophic scar.

In experiments conducted during the course of developing some embodiments of the present technology, the highest tolerable dose of Simvastatinin the rabbit ear wound model was found to be 400 μM per wound. Dose-response data were determined for Simvastatin, Lovastatin, and Pravastatin (see, e.g., Examples 1 and 2). In some embodiments of the present technology, statins (e.g., 40 μM Simvastatin, Lovastatin, or Pravastatin per wound) were surprisingly found to inhibit hypertrophic scar formation. Local administration (e.g., injection) of HMG-CoA reductase-inhibiting agents in post-surgical, traumatic, or other wounds has significant effect on hypertrophic scar formation and therefore is of benefit for wound healing and scar prevention.

### A. Wounds and Scars

In normal wound healing, the coordinated interplay between fibroblasts, vascular cells, extracellular matrix components, and epithelial cells results in a seamless progression through an inflammatory reaction, wound repair, contracture, and coverage by an epithelial barrier. However, in many subjects with a dysregulated wound microenvironment, systemic disease, or other confounding circumstances, the wound healing process becomes asynchronous resulting in an indolent ulcer (Pierce, 2001, Am. J. Pathol. 159:399). In other subjects, a loss or lack of appropriate regulatory responses to modulate cellular behaviors during healing causes an exuberant proliferative response that in itself is a problem for the subject. This is particularly true for patients prone to keloid formation or hypertrophic scar formation or in burn patients where excessive fibroblastic proliferation and collagen production result in disfiguring and disabling scar formation.

In some burns, such as deep second degree burns where dermal and hair shaft epithelial elements persist to replace lost tissues, a rich angiogenic and epithelial response is needed, but it is desirable to mitigate the fibroblastic reaction to reduce scar hypertrophy, contracture, and disfigurement. Also, suppressing the proliferative fibroblastic response in wounds is desirable in healing subjects prone to keloid formation or hypertrophic scar formation. It is also advantageous in wounds near joints or orifices to be able to promote rapid healing and coverage with epithelium while also modulating the fibroblastic response to improve tissue suppleness. Modulating the fibroblastic response in this way has the potential to provide superior clinical outcomes and reduce the need for subsequent reconstructive procedures targeted at recovering limb or other critical bodily functions.

Hypertrophic scars and keloids can be described as variations of typical wound healing. In a typical wound, anabolic and catabolic processes achieve equilibrium approximately 6-8 weeks after the original injury. At this stage, the strength of the wound is approximately 30-40% that of healthy skin. As the scar matures, the tensile strength of the scar improves as a result of progressive cross-linking of collagen fibers. At this point, the scar is usually hyperemic and it may be thickened, but it tends to subside gradually over months until a flat, white, pliable, possibly stretched, mature scar has developed. When an imbalance occurs between the anabolic and catabolic phases of the healing process, more collagen is produced than is degraded, and the scar grows in all directions. The scar is elevated above the skin and remains hyperemic. Excessive fibrous tissue is classified as either a keloid or a hypertrophic scar. Kischer and Brody declared the collagen nodule to be the identifying structural unit of hypertrophic scars and keloids (Kischer et al. (1981) Scan. Elect. Microsc. 371-376; herein incorporated by reference in its entirety). The nodule, which is absent from mature scars, contains a high density of fibroblasts and unidirectional collagen fibrils in a highly organized and distinct orientation. In addition, keloids and hypertrophic scars differ from healthy skin by a rich vasculature, high mesenchymal cell density, and thickened epidermal cell layer. Clinical differentiation of keloids from hypertrophic scars is difficult in the early phases of formation. Clinical differences become more apparent as lesions mature. The most consistent histologic difference is the presence of broad, dull, pink bundles of collagen in keloids, which are not present in hypertrophic scars.

Keloids and hypertrophic scars located at most sites are primarily of cosmetic concern; however, some keloids or hypertrophic scars can cause contractures, which may result in loss of function if overlying a joint or in significant disfigurement if located on the face. Keloids and hypertrophic scars can be both painful and pruritic and/or may cause a burning sensation.

The exact mechanisms of keloid and hypertrophic scar pathogenesis continue to be an enigma; however, the increased prevalence of keloids paralleling increased cutaneous pigmentation suggests a genetic basis or, at least, a genetic linkage. Trauma to the skin, both physical (e.g., earlobe piercing, surgery) and pathological (e.g., acne, chickenpox), is the primary cause identified for the development of keloids. The presence of foreign material, infection, hematoma, or increased skin tension can also lead to keloid or hypertrophic scar formation in susceptible individuals. Keloids and hypertrophic scars are associated genetically with HLA-B14, HLA-B21, HLA-Bw16, HLA-Bw35, HLA-DRS, HLA-DQw3, and blood group A.

### B. Statins

Statins are presently the most commonly prescribed cholesterol-lowering medications in the world. Members of this class of drugs lower cholesterol by inhibiting the enzyme HMG-CoA reductase, which is the rate-limiting enzyme of the mevalonate pathway of cholesterol synthesis. Inhibiting this enzyme in the liver results in decreased cholesterol synthesis as well as increased synthesis of LDL receptors, resulting in an increased clearance of low-density lipoprotein (LDL) from the bloodstream. The first results are clinically observable after one week of use and the effect is maximal after four to six weeks.

Natural sources of statins include fungal species that employ the molecules as inhibitors of HMG-CoA reductase as a defense response, since mevalonate is a precursor of many structurally important microbial compounds (e.g., for cell wall components such as ergosterol). The first statin agent isolated was mevastatin (ML-236B), which is produced by the fungus Penicillium citrinum. Isolation of additional statins soon followed, including Lovastatin (mevinolin, MK803), the first commercially marketed statin, which was derived from the fungus Aspergillus terreus.

In general, statins are either produced by fermentation or are synthetic. Statins include, but are not limited to, Atorvastatin (brand names Lipitor, Torvast), Cerivastatin (brand names Lipobay, Baycol), Fluvastatin (brand names Lescol, Lecol XL), Lovastatin (brand names Mevacor, altocor, Altoprey), Mevastatin (naturally occurring in organisms including, but not limited to, oyster mushrooms and Monascus purpureus), Pitavastatin (brand names Lovalo, Pitava), Pravastatin (brand names Pravachol, Selektine, Lipostat), Rosuvastatin (brand name Crestor), Simvastatin (brand names Zocor, Lipex), Simvastatin+Ezetimibe combination therapy (brand name Vytorin), Lovastatin+Niacin combination therapy (brand name Advicor), Atorystatin+Amlidipine combination therapy (brand name Caduet), and Simvastatin+Niacin combination therapy (brand name Simcor).

The LDL-lowering potency varies between statin agents. Cerivastatin is the most potent, followed by (in order of decreasing potency), rosuvastatin, atorvastatin, simvastatin, lovastatin, pravastatin, and fluvastatin (Shepherd et al. (2003) Am. J. Cardiol. 91:11C-17C; herein incorporated by reference in its entirety).

### C. Pharmaceutical Formulations and Administration

Compositions used in method embodiments of the present technology are pharmaceutically formulated for administration, e.g., topical administration or administration by local injection. Such formulations may comprise appropriate salts, buffers, solvents, dispersion media, antibacterial and antifungal agents, isotonic agents, and absorption delaying agents to render delivery of the composition in a stable manner and thus allow uptake by target tissues (e.g., epidermal tissue, scar tissue). Supplementary active ingredients may also be incorporated into the compositions. In preferred embodiments, administration is localized to a wound and/or scar site or proximal to a wound and/or scar site. In preferred embodiments, administration is via local injection or topical administration.

Dosage forms for topical or transdermal administration of statins used in some method embodiments of the present technology include powders, sprays, ointments, pastes, creams, lotions, gels, solutions, and transdermal patches. The active component may be mixed under sterile conditions with a pharmaceutically-acceptable carrier or excipient, and with any preservatives or buffers that may be important. Powders and sprays can be prepared, for example, with excipients such as lactose, talc, silicic acid, aluminum hydroxide, calcium silicates, and polyamide powder, or mixtures of these substances. The ointments, pastes, creams, and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Pharmaceutical forms for local injection of statins used in some method embodiments of the present technology include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating, such as lecithin, by the maintenance of a desired particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

### D. Dosage

Experiments conducted during the development of some method embodiments of the present technology demonstrated therapeutic effectiveness of local injection of HMG-CoA reductase inhibitors (e.g., statins) in the reduction of hypertrophic scarring (e.g., reduction in scar elevation index, reduction in excess epidermal scarring). A therapeutically effective amount of a compound in some method embodiments as described herein may vary depending upon the route of administration and dosage form. Effective amounts of compounds used in some method embodiments of the present technology typically fall in the range of about 0.001 up to 100 mg/kg/day, and more typically in the range of about 0.05 up to 10 mg/kg/day. When considered on a per-wound-site basis, a therapeutically effective amount of a compound in some method embodiments described herein typically fall within the range of 1-500 μM per wound (e.g., 1-10, 10-50, 50-100, 100-300, 300-500 μM per wound). In some preferred embodiments, a therapeutically effective amount of a compound described herein falls within the range of 20-100 μM per wound. One skilled in the art appreciates that the injection volume to be applied to a wound depends on the dose desired, the concentration of the compound as dictated by pharmaceutical formulation, and other factors (e.g., method of administration, inclusion of additionally pharmaceutically-appropriate components, subject medical history and physiology, etc.). Typically, the compound or compounds used in the technology are selected to provide a formulation that exhibits a high therapeutic index. The therapeutic index is the dose ratio between toxic and therapeutic effects, which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

### E. Methods of Combined Therapy

Some embodiments of the present technology provide methods for ameliorating or preventing abnormal wound healing (e.g., hypertrophic scars, keloids) by effectively administering a combined therapy approach.

To treat a subject using the methods of the present technology in combination therapy, one contacts a “target” tissue (e.g., wound site, wound-proximal tissue) with the compositions described herein and at least one other agent. These compositions are provided in a combined amount effective to have a therapeutic effect on the cells and tissues. This process may involve contacting the cells and tissues with multiple agents or factors at the same time. This may be achieved by contacting the cells or tissues with a single composition or pharmacological formulation that includes both agents, or by contacting the cells or tissues with two distinct compositions or formulations, at the same time, wherein one composition includes, for example, an expression construct and the other includes a therapeutic agent.

Alternatively, statin treatment may precede or follow the other agent treatment (e.g., an alternative wound-active agent) by intervals ranging from minutes to weeks. In embodiments where the agents are applied separately to the tissue, one would generally ensure that a significant period of time did not expire between the time of each delivery, such that the agents would still be able to exert an advantageously combined effect on the tissue. In such instances, it is contemplated that cells of the tissue are contacted with both modalities within about 12-24 hours of each other and, more preferably, within about 6-12 hours of each other, with a delay time of only about 12 hours being most preferred. However, in some situations, it may be desirable to extend the time period for treatment significantly such that several days (2 to 7) or several weeks (1 to 8) lapse between the respective administrations.

In some embodiments, more than one administration of the compositions provided herein or the other agent are utilized. Various combinations may be employed, where the statin composition is “A” and the other agent is “B”, as exemplified below:

A/B/A, B/A/B, B/B/A, A/A/B, B/A/A, A/B/B, B/B/B/A, B/B/A/B,
A/A/B/B, A/B/A/B, A/B/B/A, B/B/A/A, B/A/B/A, B/A/A/B, B/B/B/A,
A/A/A/B, B/A/A/A, A/B/A/A, A/A/B/A, A/B/B/B, B/A/B/B, B/B/A/B.

Other combinations are contemplated. Again, to achieve a desired therapeutic effect, both agents are delivered to a tissue in a combined amount effective to promote a desired therapeutic outcome (e.g., reduction in excessive dermal scarring, reduction in the scar elevation index).

In some embodiments of the technology, one or more compounds provided herein and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time so as to provide the desired therapeutic or prophylactic effect. In some embodiments, the compound and the additional active agents exert their effects at times that overlap. Each additional active agent can be administered separately, in any appropriate form and by any suitable route. In other embodiments, the compound is administered before, concurrently, or after administration of the additional active agents.

In various examples, the compound and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the compound and the additional active agents are administered concurrently by co-formulation.

In other examples, the compound and the additional active agents are administered at about 2 to 4 days apart, at about 4 to 6 days apart, at about 1 week part, at about 1 to 2 weeks apart, or more than 2 weeks apart.

In certain examples, a statin compound and another wound-active agent (and optionally additional active agents) are cyclically administered to a subject. Cycling therapy involves the administration of a first agent for a period of time, followed by the administration of a second agent and/or third agent for a period of time and repeating this sequential administration. Cycling therapy can provide a variety of benefits, e.g., reducing the development of resistance to one or more of the therapies, avoiding or reducing the side effects of one or more of the therapies, and/or improving the efficacy of the treatment.

In other examples, one or more compound of some embodiments of the present technology and optionally the additional active agent are administered in a cycle of less than about 3 weeks, about once every two weeks, about once every 10 days or about once every week. One cycle can comprise the administration of a statin compound and optionally the second active agent by infusion over about 90 minutes every cycle, about 1 hour every cycle, about 45 minutes every cycle, about 30 minutes every cycle, or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the compositions provided herein can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

The additional active agents can act additivety or, more typically, synergistically with the statin compound(s). In one example, one or more compound is administered concurrently with one or more second active agents in the same pharmaceutical composition. In another example, one or more statin compound is administered concurrently with one or more second active agents in separate pharmaceutical compositions. In still another example, one or more compound is administered prior to or subsequent to administration of a second active agent. The methods provided herein contemplate administration of a compound and a second active agent by the same or different routes of administration, e.g., parenteral (e.g., local injection) or topical. In certain embodiments, when the compound is administered concurrently with a second active agent that potentially produces adverse side effects including, but not limited to, toxicity, the second active agent can advantageously be administered at a dose that falls below the threshold at which the adverse side effect is elicited.

Additional wound-active agents that may be used in combination methods in some embodiments provided herein include but are not limited to corticosteroids, interferon (IFN), 5-fluorouracil (5-FU), doxorubicin (Adriamycin), bleomycin, verapamil, retinoic acid, imiquimod, tamoxifen, tacrolimus, botulinum toxin, onion extract, hydrocortisone, silicone, vitamin E, TGF-beta (TGF-beta1, TGF-beta2, TGF-beta3), VEGF inhibitors, mannose-6-phosphate inhibitors, etanercept, recombinant human interleukin (rhIL-10), proline-cis-hydroxyproline, azetidine carboxylic acid, tranilast, pentoxifylline, anti-TGF agents (e.g., decorin), and Gentian violet.

In addition, the methods provided herein may be combined with other treatment methods for wounds and/or scars (e.g., hypertrophic scars, keloids), such methods including but not limited to occlusive dressings, compression therapy, cryosurgery, excision, radiation therapy, laser therapy, and phototherapy (e.g., photodynamic therapy, UVA-1 therapy, narrowband UVB therapy, intense pulsed light (IPL)).

### EXAMPLES

The following examples are provided to demonstrate and further illustrate certain preferred embodiments and aspects of the present technology and are not to be construed as limiting the scope thereof.

### Example 1

### Evaluation of the Effects of Simvastatin on Hypertrophic Scar Formation

An established hypertrophic scar model was employed for all procedures described herein (Brown et al. (2008) Plast. Reconstr. Surg. 121:1165-1172; Kim et al. (2003) Wound Repair Regen. 11:368-372; Kryger et al. (2007) J. Am. Coll. Surg. 205:78-88; Lu et al. (2005) J. Am. Coll. Surg. 201:391-397; Morris et al. (2007) Plast. Reconstr. Surg. 100:674-681; Reid et al. (2006) Wound Repair Regen. 14:138-141; Reid et al. (2007) J. Plast. Reconstr. Aesthet. Surg. 60:64-72; Saulis et al. (2002) Plast. Reconstr. Surg. 110:177-183; each herein incorporated by reference in its entirety). A pilot study was conducted using two rabbits to evaluate the effects of Simvastatinon hypertrophic scar formation. Given the concentrations used with in vitro studies on lung fibroblasts (Watts et al. (2005) Am. J. Respir. Cell Mol. Biol. 32:290-300; Watts et al. (2006) Respir. Res. 7:88-102; each herein incorporated by reference in its entirety), concentrations up to 100 μM were used. While the present technology is not limited to any particular mechanism, and an understanding of the mechanism is not necessary to practice the present technology, it was contemplated that up to 100 μM Simvastatin per wound overwhelms the in vivo system. Example calculations of volumes and amounts of Simvastatin are presented infra:

100 μM=0.100 mmole/L×458.6 mg/mmole=45.86 mg/L

Average volume of wound=πr²×depth

π×(5 mm)²×(2 mm deep)=157 μL

157 μL (wound volume)+100 μL (injection volume)=257 μL

45.86 mg/L×257 μL=11 μg per wound˜˜100 μM per wound

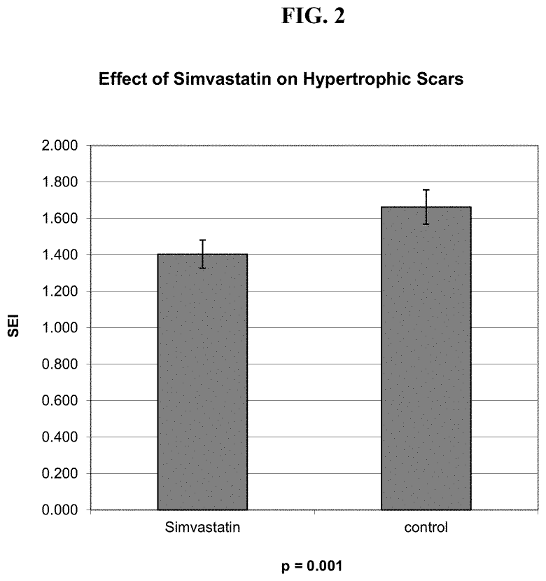

Six 10-mm wounds were made in each rabbit ear (2 rabbits total, New Zealand White), and 3 different Simvastatin doses were injected at 3 time points to test any local toxic effects that Simvastatin or vehicle (DMSO) may induce in the rabbit ears. Up to 400 μM per wound was tolerated by the rabbits. The effects of Simvastatin versus control (no treatment) were determined by analyzing the scar elevation index (SEI) for each wound (FIG. 1). Results indicated that Simvastatin causes a statistically significant decrease in SEI (p=0.001; FIG. 2). It was established that the ideal rabbit ear model uses 7-mm wounds leaving the perichondrium intact rather than 10-mm wounds. Therefore, 7-mm wounds were used for subsequent experiments (see, e.g., Example 2).

### Example 2

### Effect of Simvastatin, Lovastatin, or Pravastatin on Hypertrophic Scar Formation

Experiments were performed to analyze three dosage levels of three statins (Simvastatin, Lovastatin, or Pravastatin) on hypertrophic scar formation using the rabbit ear model described (see, e.g., Example 1 and Brown et al. (2008) Plast. Reconstr. Surg. 121:1165-1172; Kim et al. (2003) Wound Repair Regen. 11:368-372; Kryger et al. (2007) J. Am. Coll. Surg. 205:78-88; Lu et al. (2005) J. Am. Coll. Surg. 201:391-397; Morris et al. (2007) Plast. Reconstr. Surg. 100:674-681; Reid et al. (2006) Wound Repair Regen. 14:138-141; Reid et al. (2007) J. Plast. Reconstr. Aesthet. Surg. 60:64-72; Saulis et al. (2002) Plast. Reconstr. Surg. 110:177-183; each herein incorporated by reference in its entirety). A total of 28 rabbits were included in the study (New Zealand White). Wound dimensions were 7 mm. The study protocol was as follows:

### Post-Operative Day 0:

7-mm wounds were made with perichondrium left intact (6 wounds per rabbit ear). All wounds on both ears were covered with Tegaderm.

### Post-Operative Day 15: (the Point at Which the Wounds Are Completely Epithelialized)

The Tegaderms were removed. Differing dose levels (low, medium, or high doses) of Simvastatin, Lovastatin, or Pravastatin were injected in one ear and the equivalent dose of 1:1 DMSO/EtOH (vehicle) was injected in wounds on the opposite ear. Therefore, there were respective low, medium, and high control injections, depending on which group the rabbit was in.

### Post-Operative Days 20 and 25:

Injections were repeated as described above for Post-operative day 15.

### Post-Operative Day 35:

The rabbits were euthanized and wound tissue was harvested. Each wound was bisected, with half of the wound embedded in paraffin, cut, and stained with hematoxylin and eosin (H&E) to evaluate the SEI; and half flash-frozen for RNA extraction and PCR to detect levels of connective tissue growth factor (CTGF) in wound tissue.

Example dosage calculations for Simvastatin, Lovastatin, and Pravastatin are shown infra:
Average volume of wound=πr^{r}×depth
For 7 mm wound: 3.14×12.25×2=5.5 μg per wound

### Therefore, for 400 μMol Simvastatin:

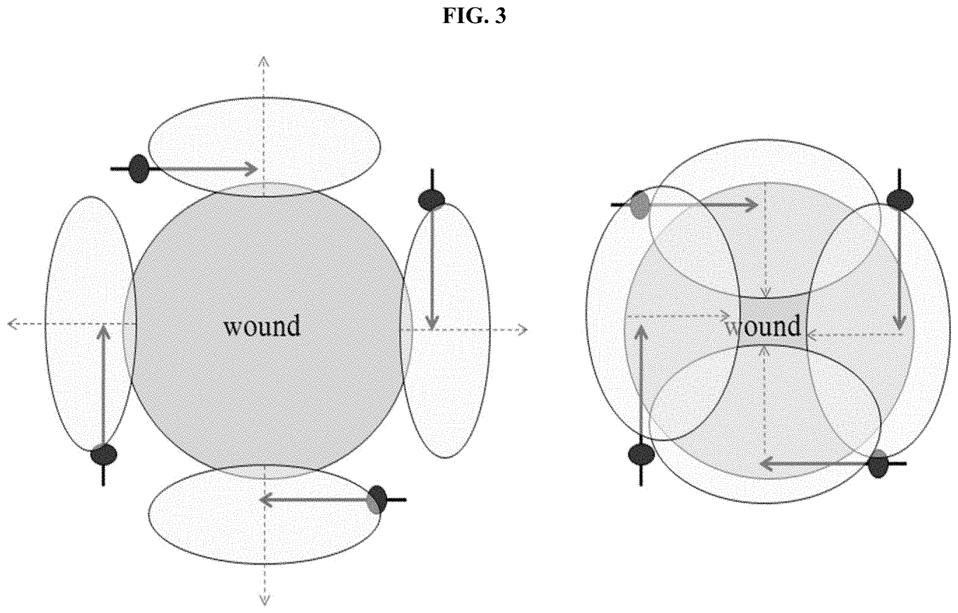

High dose: 400 μMol=22 μg/wound
Medium dose: 120 μMol=6.7 μg/wound
Low dose: 40 μMol=2.2 μg/wound

Whereas the dosages in terms of μMol were equivalent for all 3 statins, the μg dosage levels for Lovastatin and Prevastatin were double those used for Simvastatin. Injections were administered as indicated in FIG. 3. Scar Elevation Index was determined for 28 rabbits, as indicated:

### Simvastatin

40 μMol dose: 3 rabbits
120 μM dose: 2 rabbits
400 μM dose: 3 rabbits

### Lovastatin

40 μM dose: 3 rabbits
120 μM dose: 2 rabbits
400 μM dose: 3 rabbits

### Pravastatin

40 μM dose: 2 rabbits
120 μM dose: 2 rabbits
400 μM dose: 3 rabbits
Control vs. No Treatment
2 rabbits

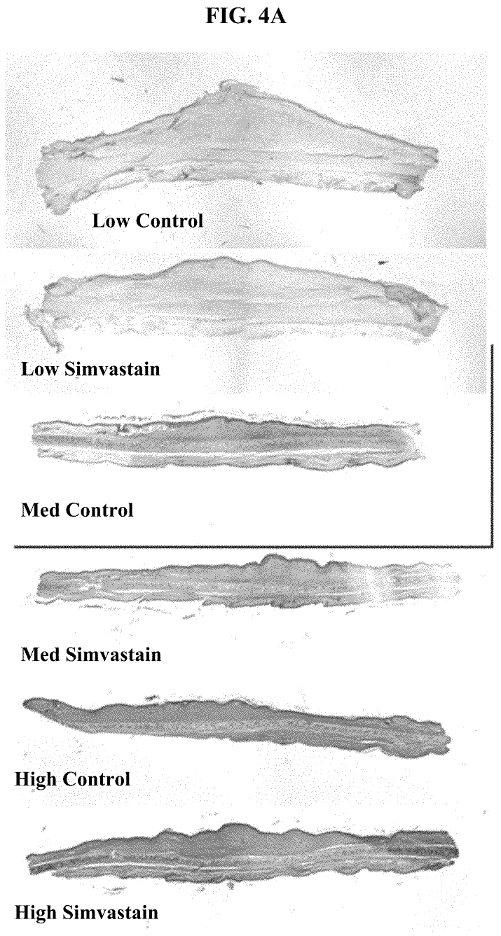

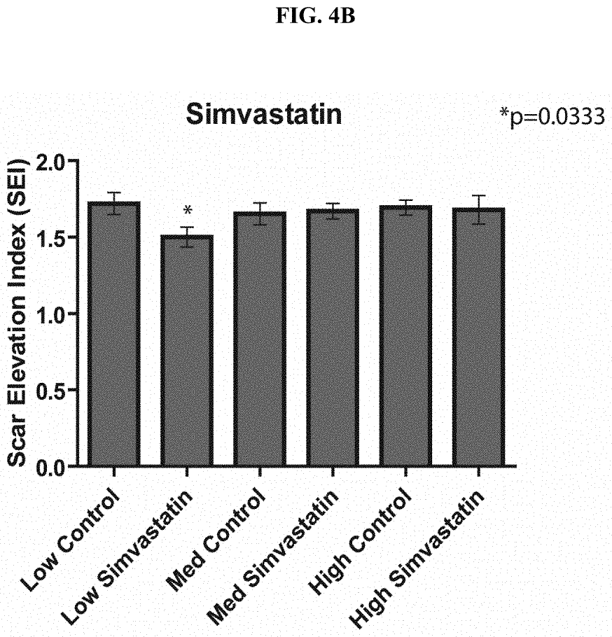

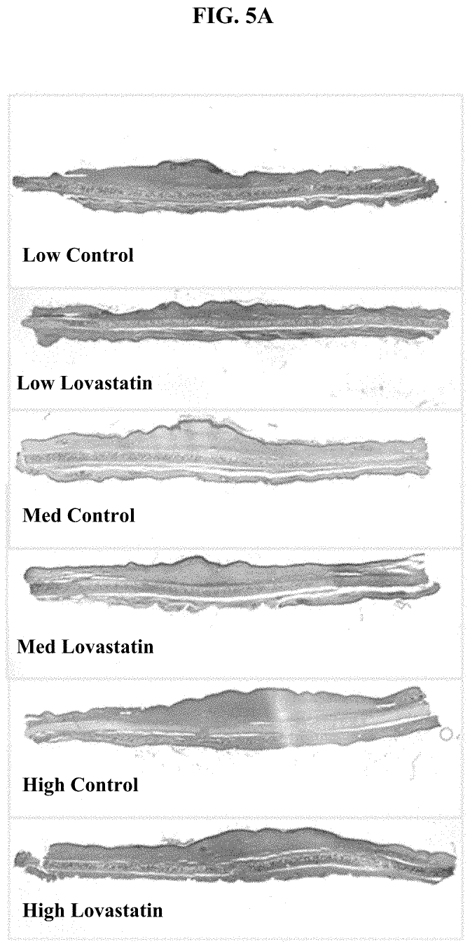

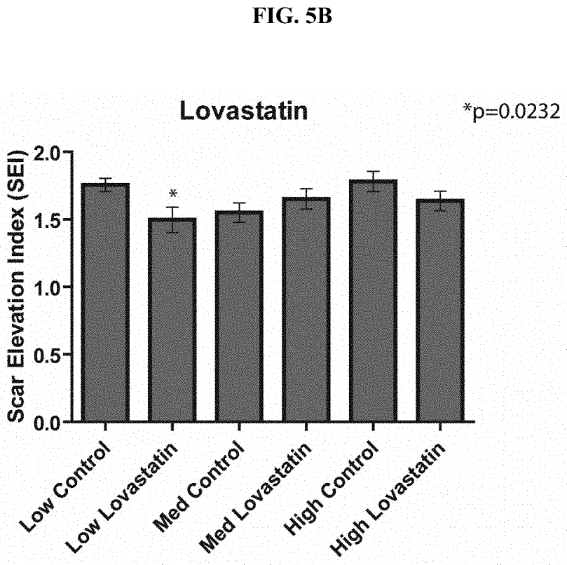

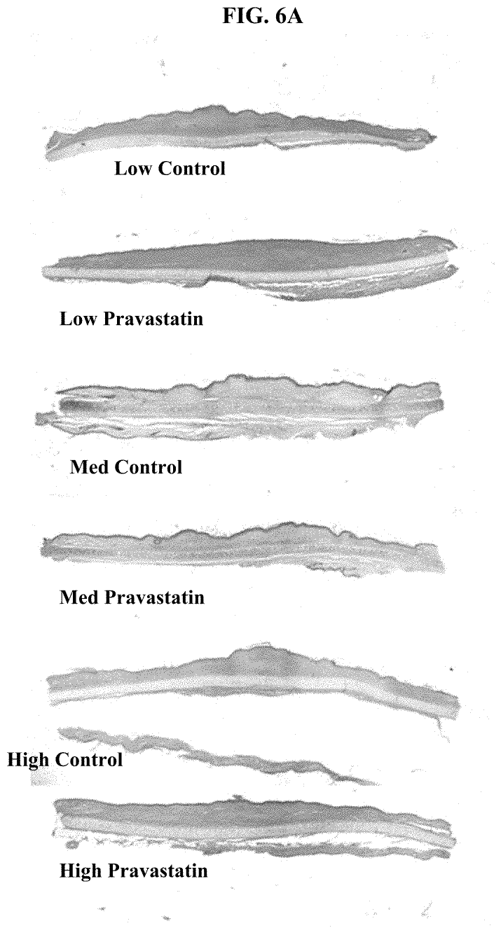

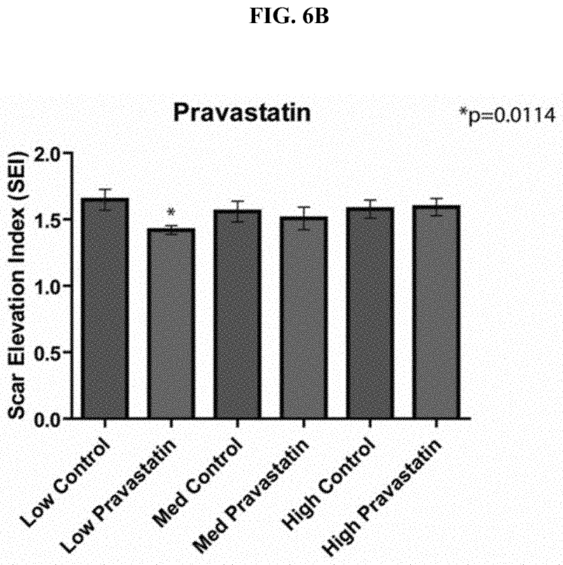

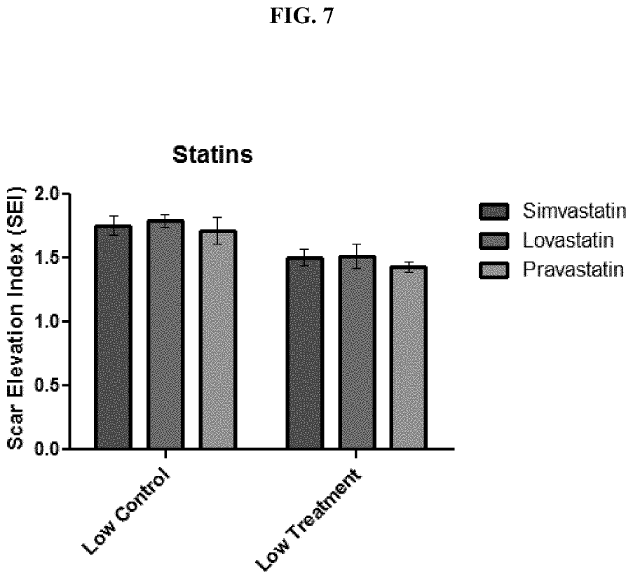

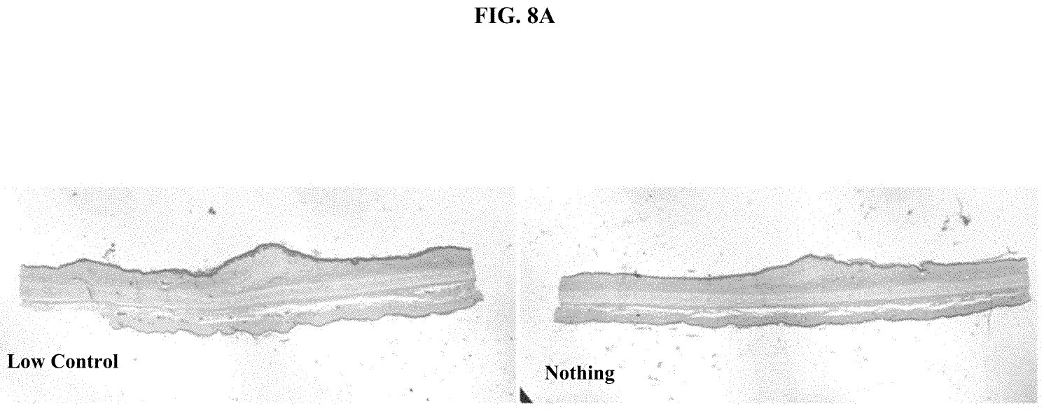

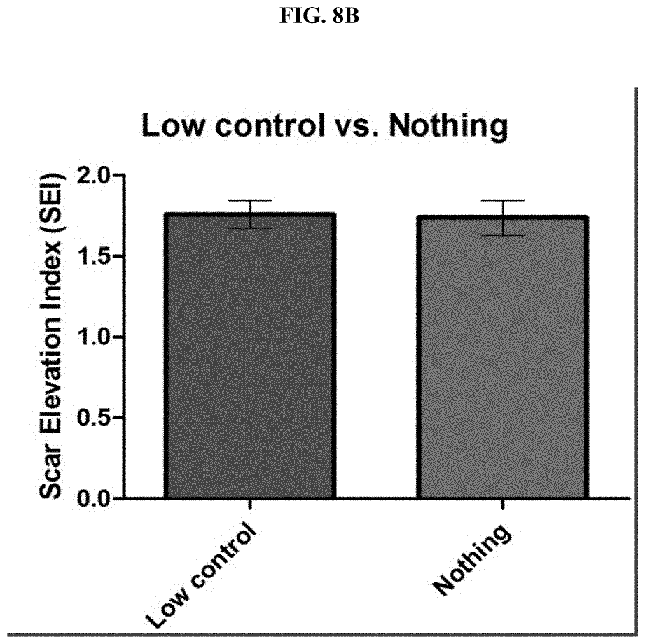

Results are shown in FIGS. 4A and 4B (Simvastatin), FIGS. 5A and 5B (Lovastatin), FIGS. 6A and 6B (Pravastatin), FIG. 7 (composite), and FIGS. 8A and 8B (controls). For all three statins, significantly lower SEI was observed at the lowest level of drug administered.

### Example 3

### Effect of Simvastatin on CTGF Expression

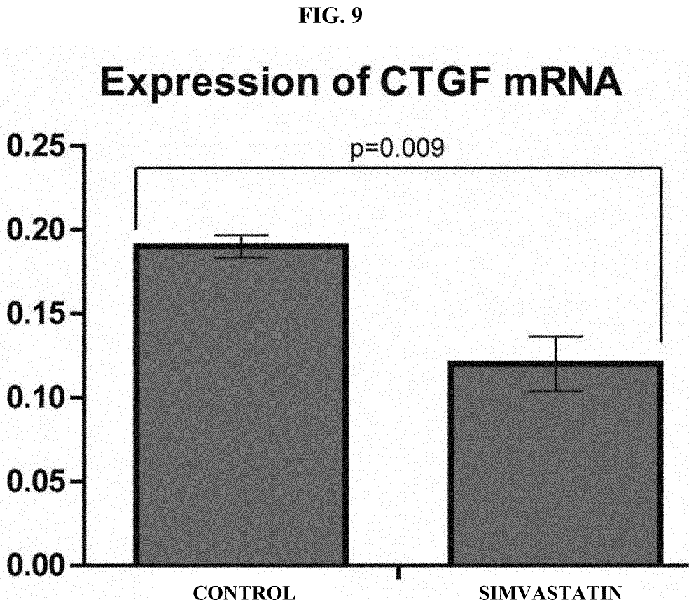

In an additional experiment, a similar protocol was conducted using only low-dose (40 μM) Simvastatin injections, administered on days 18, 19, 20, with sacrifice and harvest on day 21. Each wound was bisected, with half of the wound embedded in paraffin, cut, and stained with hematoxylin and eosin (H&E) in order to evaluate the SEI; and half flash-frozen for RNA to subsequently be extracted and PCR used to detect levels of connective tissue growth factor (CTGF) in wound tissue. PCR demonstrated down-regulation of CTGF (FIG. 9), confirming the hypothesis that CTGF plays a significant role in wound healing and that administration of Simvastatin is correlated with down-regulation of CTGF.

All publications and patents mentioned in the above specification are herein incorporated by reference. Various modifications and variations of the methods and compositions provided herein will be apparent to those skilled in the art without departing from the scope and spirit of the technology. Although the technology has been described in connection with specific preferred embodiments, it should be understood that the technology as claimed should not be unduly limited to such specific embodiments. Indeed, various modifications of the described modes for carrying out the technology that are obvious to those skilled in molecular biology, genetics, physiology, biochemistry, medical science, or related fields are intended to be within the scope of the following claims.

We claim:

1. A method for treating a subject comprising: administering a HMG-CoA reductase-inhibiting agent locally to a wound site.

2. The method of claim 1, wherein said wound site comprises scar tissue.

3. The method of claim 1, wherein said wound site does not comprise scar tissue.

4. The method of claim 1, wherein said HMG-CoA reductase-inhibiting agent comprises a statin.

5. The method of claim 4, wherein said statin is selected from the group consisting of Atorvastatin, Cerivastatin, Fluvastatin, Lovastatin, Mevastatin, Pitavastatin, Pravastatin, Rosuvastatin, and Simvastatin.

6. The method of claim 1, wherein said administering occurs by local injection.

7. The method of claim 1, further comprising administering an agent selected from the group consisting of corticosteroids, interferon, 5-fluorouracil, doxorubicin, bleomycin, verapamil, retinoic acid, imiquimod, tamoxifen, tacrolimus, botulinum toxin, onion extract, hydrocortisone, silicone, vitamin E, TGF-beta, TGF-betal, TGF-beta2, TGF-beta3, VEGF inhibitors, etanercept, mannose-6-phosphate inhibitors, recombinant human interleukin-10, proline-cis-hydroxyproline, azetidine carboxylic acid, tranilast, pentoxifylline, an anti-TGF agent, and Gentian violet.

8. The method of claim 1, further comprising administering an additional treatment selected from the group consisting of occlusive dressings, compression therapy, cryosurgery, excision, radiation therapy, laser therapy, photodynamic therapy, UVA-1 therapy, narrowband UVB therapy, and intense pulsed light therapy.

9. The method of claim 1, wherein said HMG-CoA reductase-inhibiting agent is administered at a dose ranging from 1-500 μM per wound site.

10. The method of claim 1, wherein said HMG-CoA reductase-inhibiting agent is administered at a dose of 0.1-1000 μg per wound site.

11. The method of claim 1, wherein said method results in a reduction in scar elevation index.

12. The method of claim 11, wherein said scar elevation index is lowered by at least 5%.

13. The method of claim 11, wherein said scar elevation index is lowered by at least 10%.

14. The method of claim 11, wherein said scar elevation index is lowered by at least 20%.

15. The method of claim 11, wherein said scar elevation index is lowered by at least 50%.

16. The method of claim 1, wherein said subject requires treatment to prevent hypertrophic scar formation.

17. A method of inhibiting expression of connective tissue growth factor in epidermal tissue comprising: administering a HMG-CoA reductase-inhibiting agent locally to said epidermal tissue.

18. The method of claim 17, wherein said administration occurs proximal to a wound site.

19. The method of claim 17, wherein said administration occurs at a wound site.

20. The method of claim 17, wherein said administration occurs by local injection.

21. The method of claim 17, wherein said HMG-CoA reductase-inhibiting agent is selected from the group consisting of Atorvastatin, Cerivastatin, Fluvastatin, Lovastatin, Mevastatin, Pitavastatin, Pravastatin, Rosuvastatin, and Simvastatin.
